(19) United States
(12) Patent Application Publication
(10) Pub. No.: US 2019/0059709 A1
(43) Pub. Date: Feb. 28, 2019

(54) INSERT TUBE AND ENDOSCOPE USING THE SAME

(71) Applicant: OPCOM INC., New Taipei City (TW)

(72) Inventor: Chih-Yu Ting, New Taipei City (TW)

(21) Appl. No.: 16/101,541

(22) Filed: Aug. 13, 2018

Related U.S. Application Data
(60) Provisional application No. 62/551,283, filed on Aug. 29, 2017.

(51) Int. Cl. A61B 1/04; A61B 1/018; A61B 1/00; A61B 1/005

(52) U.S. Cl. CPC A61B 1/04; A61B 1/018; A61B 1/0055; A61B 1/0052; A61B 1/00071

(57) ABSTRACT

An insert tube and an endoscope using the same are provided. The insert tube includes an instrument channel, a camera module and a distal section. The camera module includes a camera. The distal section includes a first face and a second face disposed on one end of the distal section, respectively. A first opening and a second opening are formed on the first face and the second face, respectively. A predetermined distance is a distance between the first face and the second face, and one end of the instrument channel and the camera are located at the first opening and the second opening, respectively.

### CROSS REFERENCE TO RELATED APPLICATIONS

This application claims the benefit of U.S. Provisional Application No. 62/551,283, which was filed on Aug. 29, 2017, and is incorporated herein by reference.

### BACKGROUND OF THE INVENTION

### 1. Field of the Invention

The invention relates in general to an insert tube and an endoscope using the same, and more particularly to a predetermined distance between a camera of camera module disposed in the insert tube and an instrument channel of the insert tub and an endoscope using the same.

### 2. Description of the Prior Art

Currently, an endoscope usually includes an insert tube which forms a plurality of channels therein. Therefore, if the endoscope must have many functions, the insert tube may not be reduced in accordance with the needs of the user.

Usually, the insert tube has at least one instrument channel and at least one camera channel. And the insert tube may have a distal end, and the distal end has many holes which are corresponding to the channels to receive at least one camera or at least one instrument. Similarly, the size of the distal end may not be reduced for forming many holes to receive many elements.

However, when the patient is in the endoscopic surgery, the size of the wound of the patient for inserting the endoscope may be greater than or equal to an inside diameter of the distal end. Therefore, when the inside diameter of the distal end is greater in accordance with the necessary, the size of the wound of the patient may not be reduce and burden on a patient may not be reduced.

If the size of the insert tube is reduced for reducing the burden on the patient, the number of the channels of the insert tube must be reduced or the size of the instrument channel and/or the size of the camera channel must be reduced, meanwhile, the resolution of the camera is reduced. The user may not have a good observation caused by the images captured from the camera with the low resolution and may reduce the exact determination of the operation of the surgery or cause of disease.

### SUMMARY OF THE INVENTION

The disclosure is directed to an insert tube and an endoscope using the same. A predetermined distance is a distance between a camera and an instrument channel of the insert tube.

According to one aspect of the present disclosure, an insert tube is provided. The insert tube includes an instrument channel, a camera module and a distal section. The camera module includes a camera. The distal section includes a first face and a second face disposed on one end of the distal section, respectively. A first opening and a second opening are formed on the first face and the second face, respectively. A predetermined distance is a distance between the first face and the second face, and one end of the instrument channel and the camera are located at the first opening and the second opening, respectively.

According to another aspect of the present disclosure, an endoscope is provided. The endoscope includes an insert tube, a cable and a handle. One end of the handle connects to one end of the insert tube, and the other end of the handle connects to the cable. The insert tube includes an instrument channel, a camera module and a distal section. The camera module includes a camera. The distal section includes a first face and a second face disposed on one end of the distal section, respectively. A first opening and a second opening are formed on the first face and the second face, respectively. A predetermined distance is a distance between the first face and the second face, and one end of the instrument channel and the camera are located at the first opening and the second opening, respectively.

According to one aspect of the present disclosure, an insert tube is provided. The insert tube includes an instrument channel, a camera module and a distal section. The distal section includes an extending portion disposed in one end of the distal section, and a first opening is formed in the end of the distal section and near the extending portion, wherein one end of the instrument channel and the camera are located at the first opening and the extending portion.

According to another aspect of the present disclosure, an endoscope is provided. The endoscope includes a camera module, a handle and an insert tube. One end of the handle connects to one end of the insert tube, and the other end of the handle connects to the cable. The insert tube includes an instrument channel, a camera module and a distal section. The distal section includes an extending portion disposed in one end of the distal section, and a first opening is formed in the end of the distal section and near the extending portion, wherein one end of the instrument channel and the camera are located at the first opening and the extending portion.

By the features described above, the present invention provides an insert tube and an endoscope using the same, the predetermined distance between the first face and the second face is the extending portion of the distal section, therefore, the whole size of the distal section of the insert tube may be reduced and smaller, and the resolution of the camera may be kept because of the size of the camera may not limited to the whole size of the distal section, the distal section may be inserted into and through the patient's organs and cavities easily and burden on a patient may be reduced because of the smaller size of the distal section.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a side view schematically showing a whole endoscope according to one embodiment of the present invention.

FIG. 2 is an exploded view of an insert tube according to one embodiment of the present invention.

FIG. 3 is a side view schematically showing a distal section of the insert tube according to one embodiment of the present invention.

FIG. 4 is a cross-sectional view of the distal section of the insert tube according to one embodiment of the present invention.

FIG. 5 is a cross-sectional view of the distal section of the insert tube according to another embodiment of the present invention.

### DETAILED DESCRIPTION

Preferred embodiments are disclosed below for elaborating the invention. Various lengths, such as optical focus lengths or distances, of a plurality of micro lenses are applied on a plurality of detecting pixels, such that in an autofocus process, the time consumption can be reduced, and the accuracy can be improved. The following embodiments are for the purpose of elaboration only, not for limiting the scope of protection of the invention. Besides, secondary elements are omitted in the following embodiments to highlight the technical features of the invention.

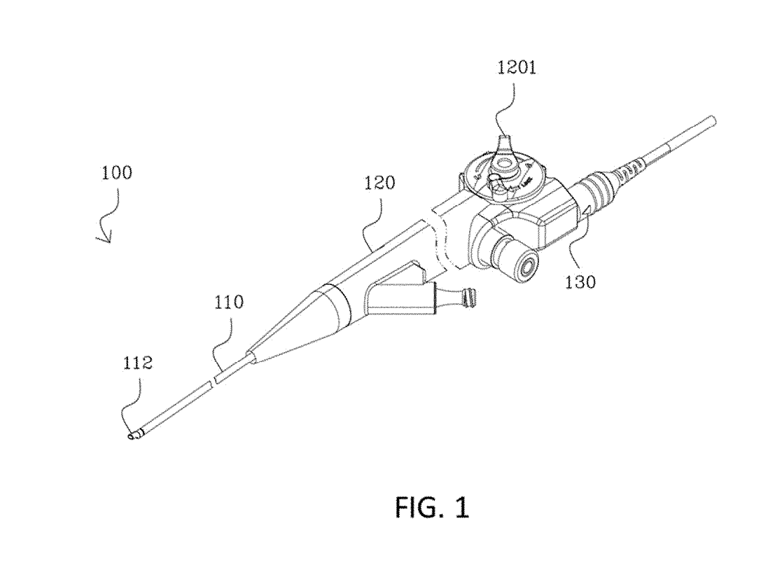

Reference is now made to FIG. 1, which is a side view schematically showing a whole endoscope according to one embodiment of the present invention. As shown in FIG. 1, the endoscope 100 includes an insert tube 110, a handle 120 and a cable 130. Wherein, one end of the handle 120 connects to one end of the insert tube 110, and the other end of the handle 120 connects to one end of the cable 130. The endoscope 100 may be a disposable endoscope or a reusable endoscope. The disposable endoscope or the reusable endoscope may be chosen in accordance with user's needs. The insert tube 110 may be a flexible tube or an inflexible tube. But it is not limit this invention.

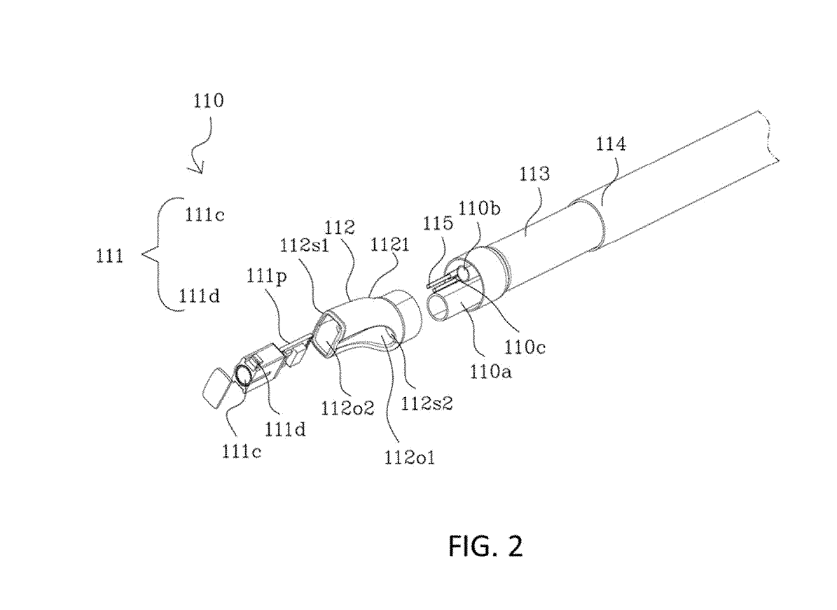

Please refer to FIG. 2, which is an exploded view of an insert tube according to one embodiment of the present invention. As shown in FIG. 1 and FIG. 2, the insert tube 110 includes an instrument channel 110a, a camera module 111, a distal section 112, a bending section 113, a catheter 114 and a control element 115. In the current embodiment, one end of the bending section 113 connects to the distal section 112 and one end of the control element 115, respectively, and the other end of the bending section 113 connects to one end of the catheter 114. The instrument channel 110a is further located in the catheter 114 and the bending section 113, in other words, the instrument channel 110a is disposed through the catheter 114 and the bending section 113. Moreover, the distal section 112, the bending section 113 and the catheter 114, or the bending section 113 and the catheter 114 are integrally formed in one piece to save a connecting element (no shown) between the bending section 113 and the catheter 114, and reduce the whole size and achieve waterproof effect.

The camera module 111 includes a camera 111c, a circuit board 111p and a wire 111a, wherein, the circuit board 111p electronically connects to the camera 111c and one end of the wire 111a, respectively. The camera module 111 of the current embodiment further includes at least one light emitter unit 111d, the light emitter unit 111d is disposed near the camera 111c. In another embodiment, the camera module 111 further includes two light emitter unit 111d are disposed in two sides of the camera 111c, respectively. A line of each light emitter unit 111d electronically connects to the circuit board 111p. The camera 111c and the light emitter unit 111d are located at the distal section 112, respectively.

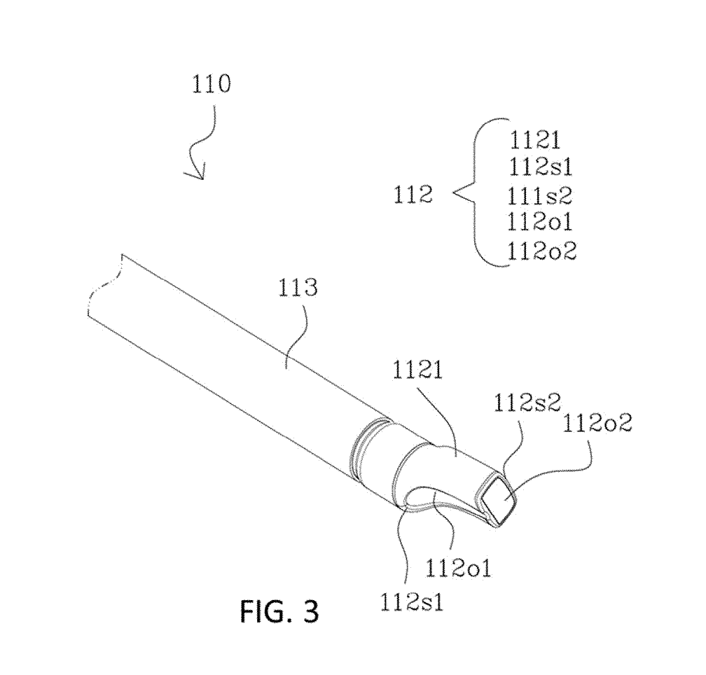

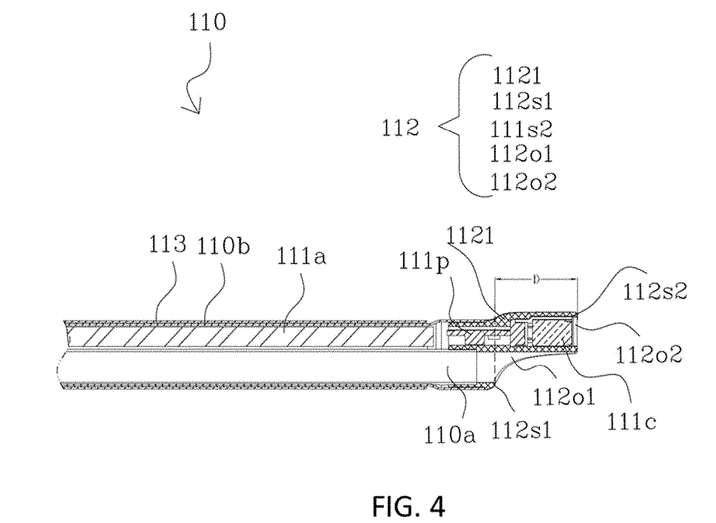

Please refer to FIG. 3, which is a side view schematically showing a distal section of the insert tube according to one embodiment of the present invention. Please refer to FIG. 4, which is a cross-sectional view of the distal section of the insert tube according to one embodiment of the present invention. As shown in FIG. 2 to FIG. 4, one end of the distal section 112 includes a first face 112s1 and a second face 112s2, a first opening 112o1 is formed on the first face 112s1, and a second opening 112o2 is formed on the second face 112s2. In the current embodiment, one end of the instrument channel 110a and the camera 111c are located in the first opening 112o1 and the second opening 112o2, respectively. The first face 122s1 and the second face 122s2 are substantially in parallel and are not in the same face.

In another alternative embodiment, the distal section 112 further includes an extending portion 1121, the extending portion 1121 is disposed in the end of the distal section 112, and the first opening 112o1 is near the extending portion 1121. The extending portion 1121 includes the second face 112s2 and a second opening 112o2. Furthermore, the first opening 112o1 and the second opening 112o2 are formed on the first face 112s1 of the end of the distal section 112 and the second face 112s2, respectively, and the second face 112s2 is further formed on one end of the extending portion 1121. The other end of the extending portion 1121 is disposed in the end of the distal section 112, and the other end of the extending portion 1121 is near the first opening 112o1 of the first face 112s1. Moreover, the extending portion 1121 is further above or below the first face 112s1. Therefore, the extending portion 1121 is located over the first opening 112o1. The end of instrument channel 110a and the camera 111c are located in the first opening 11201 and the extending portion 1121, respectively, on the other hand, the end of the instrument channel 110a and the camera 111c are located in the first opening 112o1 and the second opening 112o2 of the extending portion 1121.

Furthermore, the end of the instrument channel 110a further connects to the first opening 112o1 or the second opening 112o2, the camera 111c is also located in the first opening 112o1 or the second opening 112o2. For example, the instrument channel 110a connects to the second opening 112o2, and the camera 111c is located in the first opening 112o1. Here, the instrument channel 110a is exemplified to connect to the first opening 112o1 and the camera 111c is located in the second opening 112o2.

In the current embodiment, a distance between the first face 112s1 and the second face 112s2 of the distal section 112 of the insert tube 110 is a predetermined distance D. Furthermore, the predetermined distance D is greater than or equal to the length of the extending portion 1121. Therefore, the predetermined D between the first face 112s1 and the second face 112s2 is greater than or equal to the length of the extending portion 1121 because of the extending portion 1121 located in the end of the distal section 112.

In another current embodiment, the predetermined distance D is substantially greater than or equal to a length of the camera 111c along a long-axis direction of the camera 111c. It means that the distance between the first face 112s1 and the second face 112s2 of the distal section 112 of the insert tube 110 is the predetermined distance D, and the predetermined distance D may be greater than or equal to the length of the camera 111c and may further be greater than or equal to the length of the camera 111c along the long-axis direction of the camera 111c.

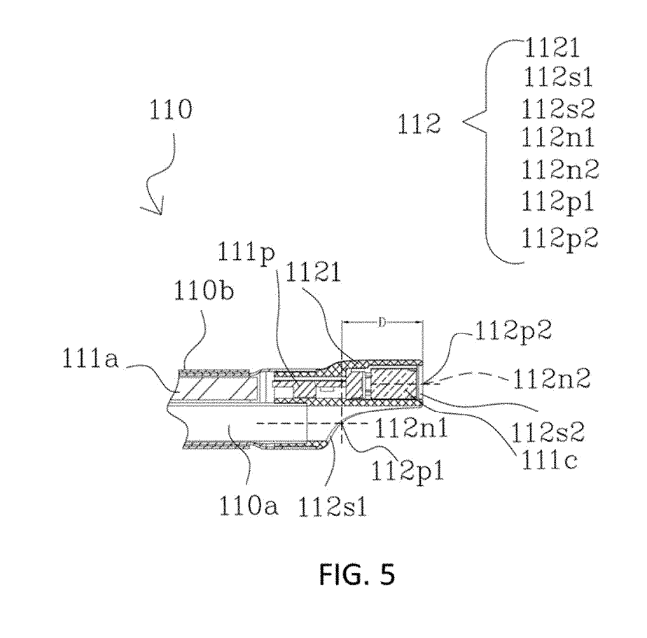

Please refer to FIG. 5, which is a cross-sectional view of the distal section of the insert tube according to another embodiment of the present invention. As shown in FIG. 5, in the current embodiment, the first face 112s1 and the second face 112s2 include a first normal line 112n1 and a second normal line 112n2, respectively. A first point 112p1 is at the intersection of the first face 112s1 and the first normal line 112n1, and a second point 112p2 is at the intersection of the second face 112s2 and the second normal line 112n2, the predetermined distance D is a distance between the first point 112p1 and the second point 112p2.

As shown in FIG. 4, in this current embodiment, a cross-sectional view of the distal section 112 forms a S-shape or an arc-shape. Furthermore, a cross-sectional view of the distal section 112 is a view along a direction determined from the end of the bending section 113 to the end of the distal section 112 and may be the S-shape or the arc-shape. Therefore, a permutation of the camera 111c and the instrument channel 110a disposed in the distal section 112 is a derangement, and the camera 111c is received in a part of the distal section 112 and the instrument channel 110a is received in the other part of the distal section 112. Therefore, a whole size of the distal section 112 may be reduced and burden on a patient caused by an endoscopic surgery may be reduced.

As shown in FIGS. 1 and 2, the catheter 114 of the insert tube 110 may form at least one channel. For example, the catheter 114 further forms a plurality of channels which means the insert tube 110 includes the channels. Therefore, the catheter 114 of the insert tube 110 includes at least one instrument channel exemplified as the instrument channel 110a, a camera channel 110b and a control channel 110c, on the other hand, the instrument channel 110a, the camera channel 110b and the control channel 110c are further formed in the catheter 114 of the insert tube 110.

In the current embodiment, the insert tube 110 further includes a first cladding unit and a second cladding unit (no shown). The first cladding unit into which the catheter 114 is inserted, the second cladding unit into which the first cladding unit is inserted. In other words, the first cladding unit may be configured to cover the outer face of the catheter 114, and the second cladding unit may be configured to cover the outer face of the first cladding unit tightly.

In the current embodiment, the first cladding unit substantially may be a braid which is a net-like tube, disposed so as to cover the outer of catheter 114. The first cladding unit may further cover and fix stably on the catheter 114 and may be provided to increase the curvature and the extensibility of the bending section 113 and the life time of the insert tube 110. The first cladding unit is, for example, a tubular net-like body formed by fibers of a metal material such as a stainless steel material, a resin material or a silicone material such as aramid fiber are braided into a net-like shape.

In another current embodiment, the second cladding unit substantially may be a tube, disposed so as to cover an outer of the first cladding unit. The second cladding unit is made of, for example, a plastic material or a silicone material. The second cladding unit may be a heat shrink tubing. When the second cladding unit covers the outer of the first cladding unit, the second cladding unit may be fixed on the first cladding unit formed by a heating process or a processing which is provided to shrink the whole second cladding unit for protecting the insert tube 110.

The handle 120 of the current embodiment includes a circuit board and a controlling unit (no shown), the circuit board and the controlling unit are located in a space formed in the handle 120. The circuit board may electronically connect to the wire 111a of the camera module 111 and a wire of the cable 130. The handle 120 further includes an operation unit 1201, such as a joystick disposed the outside of the handle 120 and connects to the controlling unit disposed in the handle 120 (not shown). And the controlling unit connects to the other end of the control element 115.

In the current embodiment, the control element 115 further includes two fixed components (not shown). The control element 115 is disposed through one of the channels of the catheter 114, such as the control channel 110c. When the operational unit 1201 of the handle 120 is operated from the user, the control element 115 may be moved relatively with the controlling unit of the handle 120, and the end of the control element 115 may control the bending section 113 of the catheter 114 fixed with the fixed components to be moved or bent.

In the current embodiment, the endoscope 100 further includes a main system (not shown), the camera module 111 is further disposed through the distal section 112 and one of channels of the catheter 114, on the other hand, the light emitter unit 111d and the circuit board 111p may be disposed or located in the distal section 112. In another current embodiment, the light emitter unit 111d and the circuit board 111p may be disposed in the camera channel (no shown). But the present invention is not limited to the embodiments mentioned above. The wire 111a of the camera module 111 may be disposed through the camera channel 110b, and the camera 111c of the camera module 111, the light emitter unit 111d and the circuit board 111p may capture images to transfer the images to the circuit board of the handle 120 in accordance with the wire 111a. The circuit board of the handle 120 transfers the images to a display of the main system of the endoscope 100 via the cable 130 to display the patient's organs and cavities.

To summarize, the predetermined distance D between the first face 112s1 and the second face 112s2 is the extending portion 1121 of the distal section 112, therefore, the camera 111c and the instrument channel 110a located in the extending portion 1121 have a shift position relationship, and the camera 111c and the instrument channel 110a are not located in the same position of the distal section 112 of the insert tube 110. The whole size of the distal section 112 of the insert tube 110 may be reduced and smaller, and the resolution of the camera 111c may be kept because of the size of the camera 111c may not limited to the whole size of the distal section 112, and the resolution of the camera 111c may apply the corresponding size and/or the resolution as necessary. The distal section 112 may be inserted into and through the patient's organs and cavities easily and burden on a patient may be reduced because of the smaller size of the distal section 112 and the cross-sectional view of the distal section 112 forms the S-shape or the arc-shape.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. An insert tube, comprising:
an instrument channel;
a camera module comprising a camera; and
a distal section comprising a first face and a second face disposed on one end of the distal section, respectively, a first opening and a second opening being formed on the first face and the second face, respectively, a predetermined distance being a distance between the first face and the second face, and one end of the instrument channel and the camera being located at the first opening and the second opening, respectively.

2. The insert tube of claim 1, wherein the insert tube further comprises a bending section and a catheter, one end of the bending section connects to the distal section, the other end of the bending section connects to one end of the catheter, and the instrument channel is disposed through the bending section and the catheter.

3. The insert tube of claim 2, wherein the catheter forms at least one channel, the camera module further comprises a circuit board and a wire, the circuit board electronically connects to the camera and one end of the wire, the circuit board is located in the distal end, and the wire is disposed through the channel.

4. The insert tube of claim 1, wherein a cross-sectional view of the distal section forms a S-shape or an arc-shape.

5. The insert tube of claim 1, wherein the predetermined distance is greater than or equal to a length of the camera along a long-axis direction of the camera.

6. The insert tube of claim 1, wherein the first face and the second face comprise the first normal line and the second normal line, respectively, and a first point is at the intersection of the first face and the first normal line, a second point is at the intersection of the second face and the second normal line, and the predetermined distance is a distance between the first point and the second point.

7. An endoscope, comprising:
the insert tube claimed in claim 1;
a cable; and
a handle, one end of the handle connecting to one end of the insert tube, and the other end of the handle connecting to the cable.

8. An insert tube, comprising:
an instrument channel;
a camera module comprising a camera; and
a distal section comprising an extending portion disposed in one end of the distal section, and a first opening being formed in the end of the distal section and near the extending portion, wherein one end of the instrument channel and the camera are located at the first opening and the extending portion.

9. The insert tube of claim 8, wherein a cross-sectional view of the distal section forms a S-shape or an arc-shape.

10. The insert tube of claim 8, wherein the insert tube further comprises a bending section and a catheter, one end of the bending section connects to the distal section, the other end of the bending section connects to one end of the catheter, and the instrument channel is disposed through the bending section and the catheter.

11. The insert tube of claim 10, wherein the catheter forms at least one channel, the camera module further comprises a circuit board and a wire, the circuit board electronically connects to the camera and one end of the wire, the circuit board is located in the distal end, and the wire is disposed through the channel.

12. The insert tube of claim 8, wherein the distal section comprises a first face and a second face, the second face is formed on one end of the extending portion, the first opening and a second opening are formed on the first face and the second face, respectively, and the camera is located at the second opening.

13. The insert tube of claim 12, wherein a predetermined distance is a distance between the first face and the second face.

14. The insert tube of claim 13, wherein the predetermined distance is greater than or equal to a length of the camera along a long-axis direction of the camera.

15. The insert tube of claim 13, wherein the first face and the second face comprise the first normal line and the second normal line, respectively, a first point is at the intersection of the first face and the first normal line, a second point is at the intersection of the second face and the second normal line, and the predetermined distance is a distance between the first point and the second point.

16. An endoscope, comprising:
the insert tube claimed in claim 8;
a cable; and
a handle, one end of the handle connecting to one end of the insert tube, and the other end of the handle connecting to the cable.
